(19) United States
(12) Patent Application Publication
(10) Pub. No.: US 2005/0187285 A1
(43) Pub. Date: Aug. 25, 2005

(54) Statins (HMG-CoA reductase inhibitors) as a novel type of immunomodulator, immunosuppressor and anti-inflammatory agent

(76) Inventor: Francois Mach, Vesenaz (CH)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/111,185

(22) Filed: Apr. 20, 2005

Related U.S. Application Data
(63) Continuation of application No. 09/664,871, filed on Sep. 19, 2000, now abandoned.

(51) Int. Cl. A61K 31/401; A61K 31/366; A61K 31/225

(52) U.S. Cl. 514/423; 514/460; 514/548

(57) ABSTRACT

The present invention relates to novel methods to operate MHC-class II mediated immunomodulation, immunosuppression and anti-inflammatory action in a subject suffering from or susceptible of suffering from a condition involving inappropriate immune response, which comprises administering to the subject at least one statin, or a functionally or structurally equivalent molecule, in an amount effective to modulate MHC class II expression in the subject.

### FIELD OF THE INVENTION

The invention relates to a new use of statins as a novel type of immunomodulator. More specifically, the invention relates to methods for achieving MHC-class II-mediated immunomodulation in a subject by administration of one or more statins. The inventors have discovered that statins affect induction of MHC-class II expression by IFN-γ and thus T cell activation. This unexpected effect provides a scientific rationale for the use of statins as novel immunomodulators, in particular as immunosuppressors, not only in organ transplantation but also in numerous other pathologies. Moreover, the role of statins in repression of T lymphocyte activation makes them very useful as anti-inflammatory agents.

### BACKGROUND OF THE INVENTION

Statins are a new family of molecules sharing the capacity to competitively inhibit the hepatic enzyme 3-hydroxy-3-methylglutaryl coenzyme A (HMG-CoA) reductase. This enzyme catalyses the rate-limiting step in the L-mevalonate pathway for cholesterol synthesis. Consequently, statins block cholesterol synthesis. They are extensively used in medical practice^{1 3}, especially in the treatment of hyperlipidaemia. This class of agent is proving to be effective for preventing heart attacks in patients with hypercholesterolaemia. Moreover, reports of several large clinical trials published during recent years have clearly shown treatment with statins to reduce cardiovascular-related morbidity and mortality in patients with and without coronary disease^{1-3,8}. Recent in vitro findings indicate that statins, beside their lipid-lowering effects, may have anti-inflammatory properties and thus may regulate important molecules in vascular biology⁹.

Statins have also been tentatively connected to the immune system. Indeed, an isolated clinical observation suggesting a beneficial effect of Pravastatin on the outcome of heart transplantation was reported by Kobashigawa and colleagues⁴. This observation was left unexplained, however, and is in apparent contrast to findings reported by McPherson et al¹⁷ according to which patients without transplants who received HMG-CoA reductase inhibitors for hypercholesterolaemia showed no effect on any immunological parameter. The possibility of interactions between some statins and the immune system led to concern amongst some specialists about potential in vivo alteration of lymphoid cell function seen with long term administration of HMG-CoA reductase inhibitors¹⁸, although no clear evidence for an interaction of this type has been provided.

The immune system is highly complex and tightly regulated, with many alternative pathways capable of compensating deficiencies in other parts of the system. There are however occasions when the immune response becomes a cause of disease or other undesirable conditions if activated. Such diseases or undesirable conditions are for example autoimmune diseases (including type I diabetes, multiple sclerosis and rheumatoid arthritis), graft rejection after transplantation, or allergy to innocuous antigens, psoriasis, chronic inflammatory diseases such as atheroslerosis, and inflammation in general. In these cases and others involving inappropriate or undesired immune response there is a clinical need for immunosuppression. The pathways leading to these undesired immune responses are numerous and in many cases are not fully elucidated. However, they often involve a common step which is activation of lymphocytes. The present invention provides a new class of agents capable of repressing class II-mediated T-lymphocyte activation and consequently capable of acting as immunomodulators and anti-inflammatory agents.

The mode of action of statins on the immune system as discovered by the present inventors will be described below, followed by a discussion of the different immune-related applications of statins and the therapeutic uses of these drugs.

Major Histocompatibility Complex molecules, coded by the HLA gene cluster in man, are involved in many aspects of immunological recognition, including interaction between different lymphoid cells, as well as between lymphocytes and antigen-presenting cells. Major Histocompatibility Complex class II (MHC class II or MHC-II) molecules are directly involved in the activation of T lymphocytes and in the control of the immune response. Although all cells express class I MHC molecules, class II expression is confined to antigen-presenting cells (APCs). These cells are potentially capable of presenting antigen to lymphocytes T-helper which control the development of an immune response. Thus the expression of MHC class II molecules is the key to antigen presentation. Only a limited number of specialized cell types express MHC class II constitutively, numerous other cells become MHC class II positive upon stimulation. The stimulation is usually induction by a cytokine, particularly by interferon gamma (IFN-γ)⁵.

Regulation of expression of MHC class II genes is highly complex and this tight control directly affects T lymphocyte activation and thus the control of the immune response This complex regulation has now been dissected in great detail, thanks to a great extent to a rare human disease of MHC class II regulation, called the Bare Lymphocyte Syndrome (or MHC class II deficiency)⁵. Four groups of patients, all with an identical clinical picture of severe primary immunodeficiency, were shown to be affected genetically in one of four distinct transacting regulatory factors, essential for MHC class II gene transcription: whereas RFX5, RFX-AP or RFX-ANK are ubiquitously expressed factors, forming a protein complex that binds to the X box of MHC class II promoters^{5,10}, CIITA (Class II TransActivator) is the general controller of MHC class II expression and its own expression is tightly regulated^{6,7}. Interestingly, expression of CIITA is controlled by several alternative promoters, operating under distinct physiological conditions¹¹. CIITA promoter I controls constitutive expression in dendritic cells, promoter III controls constitutive expression in B and T lymphocytes, while CIITA promoter IV is specifically responsible for the IFN-γ inducible expression of CIITA and thus of MHC class II¹¹. The molecular basis of inducibility of CIITA promoter IV has been elucidated in detail¹².

### SUMMARY OF THE INVENTION

In this context, the inventors have demonstrated the following properties of statins in the inhibition of induction of MHC class II expression by IFN-γ and in repression of MHC class II-mediated T cell activation:

First, statins effectively repress the induction of MHC-II expression by IFN-γ and do so in a dose-dependant manner.

Second, in the presence of L-mevalonate (which is the product of the enzyme HMG-CoA reductase, the substrate thereof being HMG-CoA), the effect of statins on MHC class II expression is abolished, indicating that it is indeed the effect of statins as HMG-CoA reductase inhibitors that mediates repression of MHC class II.

Third, repression of MHC class II expression by statins is highly specific for the inducible form of MHC-II expression and does not concern constitutive expression of MHC-II in highly specialized APCs, such as dendritic cells and B cells.

Fourth, this effect of statins is specific for MHC class II and does not concern MHC class I expression.

Fifth, pretreatment of endothelial cells with statins represses induction of MHC class II and reduces subsequent T lymphocyte activation and proliferation.

Sixth, the inhibition achieved by statins on CIITA expression is a specific inhibition of the inducible promoter IV of CIITA.

The novel effect of statins as MHC class II repressor has been observed and confirmed in a number of cell types, including primary cultures of human endothelial cells (ECs), primary human smooth muscle cells, fibroblasts and monocyte-macrophages (Mφ), as well as in established cell lines such as ThP1, melanomas and Hela cells. This effect of statins on MHC class II induction is observed with different forms of statins currently used in clinical medicine. Interestingly however, different statins exhibit quite different potency as MHC class II <<repressors>>. Of Atorvastatin, Lovastatin and Pravastatin, the most powerful MHC class II repressor is Atorvastatin. Other members of the statin family, as well as functionally or structurally related molecules, should lead to the same newly described effect on MHC class II repression.

These results on the mechanism of statin inhibition of MHC class II induction allow to conclude in favor of a selective effect of statins on the induction of expression of promoter IV of the MHC class II transactivator CIITA. Failure to allow inducible expression of MHC class II molecules on the large variety of cells that normally become MHC class II positive under the effect of IFN-γ is expected to have multiple functional consequences. These concern activation of endogenous CD4 T lymphocytes, but also recognition of MHC class II molecules by CD4 T cells in an allogenic context following organ transplantation.

### DEFINITIONS

In the context of the present invention, the following terms are defined in the following manner:

A statin is a molecule capable of acting as a competitive inhibitor of HMG-CoA reductase. Members of the statin family include both naturally occurring and synthetic molecules, for example Compactin, Atorvastatin, Pravastatin, Lovastatin, Fluvastatin, Mevastatin, Cerivastatin, Simvastatin. This list is not restrictive and new molecules belonging to this large family are regularly discovered. As discovered in the framework of the present invention, these molecules also have a second function, which is the capacity to inhibit IFN-γ-induced CIITA expression in appropriate cells. A conventional test for determining whether a given molecule is a statin or not is the inhibition of sterol synthesis, especially according to the analyzed tissues and cells^{19,20}.

A molecule which is <<chemically related or structurally equivalent>> to a statin refers to a molecule whose structure differs from that of any member of the statin family by 2 or less substitutions or by modification of chemical bonds. Examples of the structure of some statins are given in FIG. 5. Molecules which are chemically related or structurally equivalent to a statin, in accordance with the inventors, possess at least the second above-mentioned function, which is the capacity to inhibit IFN-γ-induced CIITA expression in appropriate cells. This capacity is tested using the functional assay described below in the examples.

A molecule which is <<functionally equivalent>> to a statin refers to a molecule capable of inhibiting HMG-CoA reductase. Thus at least all the molecules capable of competitively inhibiting the enzyme HMG-CoA reductase and called statins possess the required property. In addition, according to the inventors, the functionally equivalent molecules also possess the capacity to inhibit IFN-γ-induced CIITA expression in appropriate cells. Again, this capacity is tested using the functional assay described below in the examples.

An IFN-γ responsive cell is a cell having a receptor in its membrane for IFN-γ and capable of transducing a signal after binding of IFN-γ. Some cells can be induced to express MHC class II by IFN-γ. The expression of MHC class II genes is considered a secondary response to IFN-γ since a long lag period is required (24 hours for optimal response in some cases) and requires ongoing protein synthesis since cycloheximide and/or puromycin, agents that inhibit protein synthesis, abrogate IFN-γ-induced MHC class II expression.

MHC Class II molecules are heterodimeric glycoproteins that present antigen to CD4⁺ T cells, leading to T cell activation. Cells which are designated <<MHC class II positive>> express MHC class II molecules either constitutively or in response to stimulation, for example by IFN-γ, and have then MHC class II molecules inserted in their cellular membrane.

In the context of the therapeutic methods of the present invention, the following terms are defined in the following manner:

An immunomodulator is an agent whose action on the immune system leads to the immediate or delayed enhancement or reduction of the activity of at least one pathway involved in an immune response, whether this response is naturally occurring or artificially triggered, whether this response takes place as part of innate immune system or adaptive immune system or the both. An MHC Class II-mediated immunomodulator is an immunomodulator whose key action on the immune system involves molecules of MHC class II.

An immunosuppressor is an agent which action on the immune system leads to the immediate or delayed reduction of the activity of at least one pathway involved in an immune response, whether this response is naturally occurring or artificially triggered, whether this response takes place as part of innate immune system or adaptive immune system or the both. An MHC Class II-mediated immunosuppressor is an immunosuppressor whose key action on the immune system involves molecules of MHC class II.

An anti-inflammatory agent is an agent capable of reducing or inhibiting, partially or totally, immediately or after a delay, inflammation or one of its manifestations, for example migration of leucocytes by chemotaxis. An MHC Class II-mediated anti-inflammatory agent is an anti-inflammatory agent whose key action on the immune system involves molecules of MHC class II.

A detrimental immune response is an immune response which is painful or prejudicial to the health of a patient on a long or short-term basis. Immune reactions against self molecules or tissues, or against xenografted tissues or organs are examples of detrimental immune responses. immunosuppression (or immunomodulation) becomes clinically desirable in cases where the immune system acts detrimentally to the health of a patient or is feared to do so, the shut down or down-regulation of the immune response being then considered as useful by the physician for the health of the patient. Such conditions can be encountered after an organ transplantation for enhancing tolerance to the graft. Another example is autoimmune disease, including type I diabetes, multiple sclerosis and rheumatoid arthritis. Cases in which immunosuppression is clinically required are not limited to those cited but further include psoriasis and other pathologies. Moreover, immunosuppression also includes prevention of undesirable immune reactions, for example before transplantation.

A transplantation concerns organ or tissue, such as heart, kidney or skin.

### BRIEF DESCRIPTION OF THE DRAWINGS

The invention may be further illustrated by reference to the accompanying drawings wherein:

FIG.1 is a series of graph panels showing that statins decreased IFN-γ induced MHC class II protein expression on human endothelial cells and macrophages. FIGS. 1a to 1f are graphs showing flow cytometric analyses for MHC class II proteins (a-e) and MHC class I (f). FIG. 1a shows flow cytometric analysis achieved on human vascular endothelial cells (ECs) treated with IFN-γ (500 U/ml, 48 hrs) alone (bold line), or with Atorvastatin 10 μM (left dotted line), Lovastatin 10 μM (bold dotted line), or Pravastatin 20 μM (right dotted line). FIG. 1b shows flow cytometric analysis achieved on ECs treated with IFN-γ (500 U/ml, 48 hrs) alone (bold line), or with Atorvastatin 40 nM, 0.2 μM, 2 μM, or 10 μM (from right to left dotted lines, respectively). FIG. 1c shows flow cytometric analysis achieved on ECs treated with IFN-γ alone (500 U/ml, 48 hrs) (bold line), or with Atorvastatin (10 μM) and L-mevalonate (100 μM) (dotted line). FIG. 1d shows flow cytometric analysis achieved on human dendritic cells (DC) under control conditions or created with Atorvastatin 10 μM (dotted line). FIG. 1e shows flow cytometric analysis achieved on the human cell line Ragi under control conditions or treated with Atorvastatin (10 μM, 48 hrs) (dotted line). FIG. 1f shows flow cytometric analysis achieved on ECs treated with IFN-γ (500 U/ml, 48 hrs) alone (bold line), or with Atorvastatin 10 μM (dotted line). For all panels, solid histograms represent MHC class II (a-e) or MHC class I (f) expression under unstimulated conditions. Each panel is a histogram representing cell numbers (y axis) vs. log fluorescence intensity (x axis) for 30,000 viable cells. Similar results were obtained in independent experiments with ECs and DCs from five different donors.

FIG. 1g is a graph showing fluorescence analysis (expressed as relative intensity) for MHC class II expression on human macrophages (1) are cells under unstimulated conditions, (2), (3), (4) and (5) are cells treated with IFN-γ alone (500 U/ml, 48 hrs), or with Atorvastatin (10 μM). Lovastatin (10 μM) or Pravastatin (20 μM), respectively. (6) are cells treated with IFN-γ (500 U/ml, 48 hrs) and stained with secondary antibody only (negative control). Similar results were obtained in separate experiments using macrophages from three different donors.

FIG. 2 is the association of a blot and its graphic representation showing that the effect of statins on IFN-γ induced MHC class II expression is mediated by the transactivator CIITA.

FIG. 2a is a reproduction of an RNAse protection assay (RPA) for MHC class II (DR-α) and FIG. 2b is a reproduction of an RNAse protection assay (RPA) for CIITA. Human vascular endothelial cells unstimulated (1), treated with IFN-γ (500 U/ml, 12 hrs) alone (2), or with Atorvastatin (10 μM) (3), Lovastatin (10 μM) (4), Pravastatin (20 μM (5), or Atorvastatin (10 μM) and L-mevalonate (100 μM) (6). GAPDH was used as a control for RNA loading. Quantification of RPA blots is expressed as the ratio of DR-α/GAPDH and CIITA/GAPDH signal for each sample. Similar results were obtained in independent experiments with ECs from four different donors. *p<0.001, **p<0.02 compared to IFN-γ treated cells (2), ***p<0.001 compared to IFN-γ/Atorvastatin treated cells (3).

FIG. 3 is a comparison of two different functional consequences of inhibition of MHC class II antigens by statins on T lymphocyte activation.
the first consequence is shown by means of the histogram representing [³H]Thymidine incorporation measured in allogenic T lymphocytes exposed (5 days) to human ECs (solid bars) or human Mφ (open bars) or pretreated during 48 hrs with IFN-γ (500 U/mL) alone (1,3), or IFN-γ (500 U/mL) with Atorvastatine (10 μM) (2,4) Similar results were obtained in independent experiments with Mφ or ECs from three different donors. *p<0.02 compared to IFN-γ treated cells.
the second consequence is shown by means of the histogram representing IL-2 release measured by ELISA in supernatants of allogenic T lymphocytes exposed (48 hrs) to human ECs (solid bars) or Mφ (open bars) pretreated 48 hrs with IFN-γ (500 U/mL) alone (1,3), or IFN-γ (500 U/mL) with Atorvastatin (10 μM) (2,4). Similar results were obtained in independent experiments with Mφ or ECs from four different donors. **p<0.01 compared to IFN-γ treated cells.

FIG. 4 is a combination of a graph and an electrophoretic gel showing that statins specifically decreased the expression of promoter IV of the transactivator CIITA on a transcriptional level.

FIG. 4a is a reproduction of an RNAse protection assay (RPA) for exon 1 of the promoter IV-specific form of CIITA (pIV-CIITA). Human vascular endothelial cells (ECs) unstimulated (1), treated with IFN-γ (500 U/ml, 12 hrs) alone (2), or with Atorvastatin (10 μM) (3), Lovastatin (10 μM) (4), Pravastatin (20 μM ) (5), or Atorvastatin (10 μM) and L-mevalonate (100 μM) (6). GAPDH was used as a control for RNA loading. Quantification of RPA blots is expressed as the ratio of pIV-CIITA/GAPDH signal for each sample. Similar results were obtained in independent experiments with ECs from three different donors. *p<0.001, **p<0.02 compared to IFN-γ treated cells (2), ***p<0.001 compared to IFN-γ/Atorvastatin treated cells (3). FIG. 4b is a graph representing a densitometric analysis of RPA from actinomycin D (Act D) studies showing the effects of Atorvastatin on pIV-CIITA mRNA levels. ECs were pretreated with IFN-γ (500 U/ml, 12 hrs), and then Act D (10 μg/ml) was added alone or with Atorvastatin (10 μM) and RNA analyzed at different time points. Band intensities or pIV-CIITA/GAPDH mRNA ratio were plotted as a semi-log function of time (hours). Data represent mean±SEM of separate experiments with cells from three different donors. FIG. 4c is a blot representing a Western blots analysis (40 μg protein/lane) of ECs treated with IFN-γ (500 U/ml) in the absence or presence of Lovastatin (10 μM) (Lova). Samples were analyzed for the phosphorylated form of Stat1-α (p Stat1-α) at different periods of time (minutes). Actin was used as a control for protein loading. Blots are representative of different experiments obtained with cells from four different donors.

FIG. 5 is a representation of the chemical structure of some commercially available statins. FIG. 5a is a chemical representation of Atorvastatin and Lovastatin. FIG. 5b is a chemical representation of Pravastatin sodium and Fluvastatin. FIG. 5c is a chemical representation of Mevastatin and Simvastatin

### DETAILED DESCRIPTION OF EMBODIMENTS OF THE INVENTION

A first aspect of the invention involves the exploitation of the molecular implication of statins in IFN-γ-mediated cell responses.

According to one embodiment of this first aspect, statins can be used in a process to regulate the IFN-γ-induced CIITA expression in IFN-γ responsive cells. This process is implemented by contacting an IFN-γ responsive cell with at least one statin. A consequence of this regulation is the possibility to regulate CIITA-dependant intra- and intercellular events. The role of CIITA being crucial in the cell, particularly for the expression of MHC class II molecules, acting on this important transactivator is a unique way to interfere with MHC class II transcription, expression and thus presentation to T lymphocytes. Similarly, repression of CIITA expression leads to the repression of T lymphocyte activation and proliferation. This leads in turn, at least partially, to the inhibition of all depending intercellular events characterizing the complex cascade of the immune response.

The process described above can be carried out either in vivo or in vitro.

For this process of regulation of IFN-γ-induced CIITA expression, molecules other than statins can be used provided they are chemically related to at least one statin and/or functionally equivalent thereto. In a preferred embodiment, the used statin is Compactin, Atorvastatin, Lovastatin, Pravastatin, Fluvastatin, Mevastatin, Cerivastatin or Simvastatin.

Among IFN-γ responsive cells are cells which become APC (Antigen Presenting Cells) upon induction by IFN-γ. These particular cells, called <<facultative APCs>>, are able to become MHC class II positive i.e. displaying MHC class II molecules on their surface if suitably stimulated. Such cells can be primary human endothelial cells, primary human smooth muscle cells, fibroblasts, monocytes-macrophages, cells of the central nervous system, ThP1, melanomas or Hela cells.

As the statins' action on stimulated CIITA expression is both dose-dependant and dependant of the type of statin, this process of contacting a cell with a particular member of the statin family at a particular dose provides a useful opportunity to control quantitatively the CIITA-expression and to set it at a given level. The relation between CIITA expression and level of MHC class II mRNA being linear, this quantitative control over expression of CIITA is transposable to MHC class II transcription and translation, i.e. MHC class II expression.

In the process of regulation of IFN-γ-induced CIITA expression described above, the regulation of IFN-γ-induced CIITA expression is preferably an inhibition or a reduction of this expression.

In a preferred mode of action of statins, or functional or structural derivatives, the regulation of IFN-γ-induced CIITA expression is solely achieved by inhibition of the CIITA inducible promoter IV. By “solely achieved” is meant that the statins have no effect, or substantially no effect, on the constitutive expression of CIITA, namely expression regulated by promoters I and III¹¹.

As mentioned above, it is surprisingly the effect of statins as HMG-CoA reductase inhibitors that mediates repression of MHC class II by inhibition of CIITA. Indeed providing the cell with L-mevalonate, which is the product of HMG-CoA reductase, abolishes inhibition by statins. The process of the invention has thus the property that the regulation is reversible at least partially, and preferably fully, by addition of L-mevalonate.

According to a further embodiment of this first aspect, the invention also concerns a screening method, more particularly a method for identifying molecules capable of inhibiting IFN-γ induced CIITA expression, this inhibition being at least partially reversible by addition of L-mevalonate. This method is carried out by contacting a cell which is IFN-γ responsive with a candidate inhibitory molecule and with IFN-γ. In a second step of the method, inhibition or absence of MHC class II expression in presence of the candidate molecule is detected. The next step is to contact the cell with L-mevalonate and to detect a total or partial reversal of the inhibitory effect.

Inhibition of IFN-γ induced CIITA expression at least partially by acting on the HMG-CoA reductase is an unexpected effect with significant clinical potential; molecules capable of effecting this can be identified by screening as described. The tested property is the ability to inhibit IFN-γ induced CIITA expression in at least partially reversible manner by addition of L-mevalonate. The cells used for this test must be responsive to stimulation by IFN-γ, preferred cells for this purpose are endothelial cells. IFN-γ and the potential inhibitor molecule are contacted with the cells; the detection of MHC class II expression is then carried out. In particular, this step can be accomplished by incubating the cells with for example fluorophore-conjugated specific antibody and then testing by flow cytometry. The skilled man will be aware of other classical ways to detect MHC-class II expression, for example by performing mixed lymphocytes reaction (allogenic T lymphocytes incubated with IFN-γ and candidate molecule-pretreated human endothelial cells) and assaying T cell proliferation. If the candidate molecule appears to be an efficient inhibitor, the additional property of reversibility is tested in a further step which comprises the addition of L-mevalonate to the previous cell culture and detection of a total or partial reversal of the inhibitory effect. This means that expression of MHC class II molecules is at least partially restored. Methods to assay this expression are the same as above. This method also provides a test for identifying functional equivalents of statins.

Implementation of this screening method leads to the selection of inhibitors of CIITA expression which can be then used as such. Following the mode of selection, their action on CIITA is at least partially reversible by addition of L-mevalonate. Inhibitors found according to this screening method may be useful as medicaments having immunosuppressive and anti-inflammatory effects or for example in fundamental biology to determine how L-mevalonate derivatives interfere in stimulation by interferon γ.

A second aspect of the invention concerns therapeutic methods exploiting the molecular effect of statins. The novel effect of statins as an effective MHC class II repressor and more particularly the mechanism of this effect via repression of promoter IV of the MHC-II transactivator CIITA provides a firm scientific rationale for the use of this drug as an immunosuppressor in organ transplantation. It also suggests numerous other practical clinical applications of statins as novel immunomodulators, in particular in diseases where aberrant expression of MHC class II and/or aberrant activation of CD4 T lymphocytes are implicated. Beyond organ transplantation, this ranges from various autoimmune diseases (including type I diabetes, multiple sclerosis and rheumatoid arthritis) to psoriasis and chronic inflammatory diseases such as atherosclerosis. The fact that statins are well-tolerated drugs may qualify them as a welcome addition to the limited current arsenal of immunosuppressive agents.

Specifically, in a first embodiment, the invention concerns a method to achieve immunomodulation in a subject in need of such treatment, this immunomodulation being mediated via MHC class II. A subject, for example a mammal, is likely to be treated by this method if he is suffering from a condition involving inappropriate immune response or if he is susceptible of suffering from it. The method comprises administering to the subject at least one statin, or a functionally or structurally equivalent molecule, in an amount effective to modulate MHC class II expression in the subject. The modulation may begin to occur immediately on administration of the statin, or may become effective within a few hours, e.g. 8 to 48 hours of administration.

In a second embodiment, the invention concerns a method to achieve immunosuppression in a mammal in need of such treatment, this immunosuppression being mediated via the MHC class II. In a preferred variant the repression is the result of repression of T lymphocyte activation. A mammal is likely to be treated by this second method if he is suffering from a condition involving detrimental immune response or if he is susceptible to suffer from it. The method comprises administering to the mammal at least one statin, or a functionally or structurally equivalent molecule, in an amount effective to suppress MHC class II expression in the subject. The suppression may begin to occur immediately on administration of the statin, or may become effective within a few hours, e.g. 8 to 48 hours of administration.

In a third embodiment, the invention concerns a method exploiting the major role of MHC class II expression in inflammation process in general i.e. a method to achieve MHC-class II mediated anti-inflammatory effect in a mammal in need of such treatment. A mammal is likely to be treated by this second method if he is suffering from a condition involving detrimental immune response or if he is susceptible to suffer from it. The method comprises administering to the mammal at least one statin, or a functionally or structurally equivalent molecule, in an amount effective to suppress MHC class II expression in the subject.

The subject treated by anyone of the three mentioned methods is preferably a human. The following properties or applications of these methods will essentially be described for humans although they may also be applied to non-human mammals, for example apes, monkeys, dogs, mice, etc. . . . The invention therefore can also be used in a veterinarian context.

A patient population susceptible of being treated by methods of the present invention includes patients who in addition to suffering from a condition involving inappropriate or detrimental immune response, may also suffer from hypercholesterolaemia, or from problems in the metabolism of lipids, particularly LDL (low-density lipoproteins), involving high levels of certain lipids. A particularly preferred group of subjects likely to be treated by one of the three methods is a subject who does not suffer from hypercholesterolaemia, irrespective of whether he has or not other risk factors for heart disease and stroke. By hypercholesterolaemia, it is meant LDL-cholesterol levels above 220 mg/dL, preferably above 190 mg/mL, after diet. In cases where a patient presents risk factors for heart disease or stroke, the ‘threshold’ level beyond which hypercholesterolaemia is considered to occur can be lower, for example down to 160 mg/dL, even down to 130 mg/dL.

The inhibition by statins of MHC class II expression is specific for IFN-γ-induced condition. This specificity is very advantageous since the immune system as a whole is not disturbed by statins. This characteristic of the treatment of the invention is of great interest since the patient under treatment is still able to fight opportunistic infections.

The methods are particularly well suited when the subject is suffering from a condition which involves IFN-γ inducible CIITA expression. Some autoimmune diseases are known to involve inappropriate IFN-γ release leading to CIITA expression in cells which do not normally express CIITA. It is for this reason that autoimmune diseases in general are particularly preferred conditions from which the subject is suffering.

Diseases which can be considered as autoimmune, are numerous. The three methods of the invention (i.e. immunomodulation, immunosuppression and regulation of inflammation) are particularly susceptible to be effective on type I diabetes, multiple sclerosis and rheumatoid arthritis.

Another appropriate condition for the application of one of the three methods, but particularly the immunosuppressive one, is that arising from an organ or tissue transplantation. In such an operation, the total immunological compatibility between the subject (i.e. the graft recipient) and the graft donor is almost impossible unless it is an autograft. Cells of the recipient, detecting the presence of non-self cells, are likely to kill those cells leading to the rejection of the graft. Improvement of the tolerance of the recipient is needed and can be accomplished by means of the immunosuppressive method described above.

The methods of the invention can be used in a preventive manner if a detrimental immune response is likely to arise. This is particularly convenient in the case of transplantation where the detrimental immune response is known to be triggered by the graft. Increased tolerance must be achieved before the transplantation and is an important part of the operation.

Other conditions which may be treated by the methods of the invention are psoriasis and inflammation in general or chronic inflammatory diseases, such as atherosclosis.

The methods of the invention are particularly well suited for a topical application in dermatology. The application can be localized directly on the site of inflammation. For this type of application, the statins, or their structural or functional derivatives, are administered in the form of a cream, a spray, a lotion, an ointment or a powder, on the skin where the inflammation occurs. This way of administering statin is useful in the local treatment of psoriasis, eczema and other skin inflammation.

The statin used to carry out one of the methods is preferably Compactin, Atorvastatin, Lovastatin, Pravastatin, Fluvastatin, Mevastatin, Cerivastatin, or Simvastatin.

Since the lipid lowering effect of the currently used statins mentioned above can be, under certain circumstances, an inopportune effect, it would be advantageous in these circumstances to benefit from an immunomodulatory, immunosuppressive or anti-inflammatory effect of statins, without the lipid-lowering effect. The three methods of the invention are then preferably carried out with a statin, or a functional or structural derivative, having an immunomodulatory effect without a lipid-lowering effect.

The methods can be part of a more general treatment of the subject or can be accompanied by a different treatment. In this case, the statin or derivative can be administered with or without other immunosuppressive drugs. In cases where other immunosuppressive drugs are administered, the statin and the other immunosuppressive drugs may be administered separately, simultaneously or sequentially. In a particular case, the statin is administered in the absence of any other immunosuppressive agents, in particular the statin is not administered in combination with cyclosporin A or cyclophosphamide.

In each method, depending on the chosen statin, or structurally or functionally equivalent derivative, the amount given to the subject must be appropriate, particularly effective to specifically modulate IFN-γ inducible MHC class II expression.

As for every drug, the dosage is an important part of the success of the treatment and the health of the patient. The degree of efficiency as immunomodulator, immunosuppressor or anti-inflammatory agent depends on the statin or derivative. An appropriate amount is comprised for example between 10 and 60 mg per day, preferably between 20 and 40 mg per day for currently used statins. It is envisaged that more effective statins may be discovered in the future, these molecules will thus be administered to the subject in smaller quantities. In every case, in the specified range, the physician has to determine the best dosage for a given patient, according to his sex, age, weight, pathological state and other parameters.

In the context of the three methods of the invention, the administration mode comprises intralesional, intraperitoneal, intramuscular or intravenous injection; infusion; or topical, nasal, oral, ocular or otic delivery. A particularly convenient frequency for the administration of statin or derivative once a day.

Since statins play a role in immune response, they can be used as immunosuppressors, immunomodulators or anti-inflammatory agents for the manufacture of a medicament for use in the treatment of a condition involving aberrant, undesirable or detrimental expression of MHC class II. Statins can be replaced by structurally or functionally equivalent molecules.

### EXAMPLES

### Materials and Methods

Reagents. Human recombinant IFN-γ was obtained from Endogen (Cambridge, Mass.). The three statins used in these studies [Atorvastatin, (Parke Davis); Lovastatin (Merck Sharp and Dohme); and Pravastatin (Bristol-Myers Squibb)] are commercially available and were obtained from commercial sources. Mouse anti-human MHC class II and MHC class I fluorescein isothiocyanate-conjugated (FITC) and unconjugated monoclonal antibodies were purchased from Pharmingen (San Diego, Calif.). Cycloheximide, actinomycin and L-mevalonate were purchased from Sigma (St. Louis, Mo.).

Cell isolation and culture. Human vascular endothelial cells (ECs) were isolated from saphenous veins by collagenase treatment (Worthington Biochemicals, Freehold, N.J.), and cultured in dishes coated with gelatin (Difco, Liverpool, England) as described elsewhere¹⁵. Cells were maintained in medium 199 (M199; BioWhittaker, Wokingham, England) supplemented with 100 U/ml penicillin/streptomycin (BioWhittaker), 5% FCS (Gibco, Basel, Switzerland), 100 μg/ml heparin (Sigma) and 50 μg/ml ECGF (endothelial cell growth factor; Pel-Freez Biological, Rogers, Ak.). Culture media and FCS contained less than 40 pg LPS/ml as determined by chromogenic Limulus amoebocyte-assay analysis (QLC-1000; BioWhittaker). Endothelial cells were >99% CD31 positive as characterized by flow cytometry and were used at passages 2-4 for all experiments.

Monocytes were isolated from freshly prepared human peripheral blood mononuclear cells obtained from leukopacs of healthy donors following Ficoll-Hypaque gradient and subsequent adherence to plastic culture flasks (90 min., 37° C.) Monocytes were cultured in RPMI 1640 medium (BioWhittaker) containing 10% FCS for 10 days¹⁵. Macrophages derived from monocytes were >98% CD64 positive as determined by flow cytometry.

The human Raji cell line (Epstein-Barr virus (EBV)-positive Burkitt lymphoma cell line) obtained from American Type Culture Collection (Rockville, Md.) and the human dendritic cells obtained as described¹⁶ were grown in RPMI-1640 medium containing 10% FCS.

Flow cytometry. Cells were incubated with FITC-conjugated specific antibody (60 min, 4° C.) and analyzed in a Becton Dickinson FACScan flow cytometer as described¹⁵. At least 100.000 viable cells were analyzed per condition. Data were analyzed using CELLQUEST software (Becton Dickinson).

Immunolabeling. Cells grown on coverslips were fixed for 5 min with methanol at −20° C. The coverslips were rinsed and incubated successively with 0.2% Triton X-100 in PBS for 1 hour, 0.5 M NH₄Cl in PBS for 15 min and PBS supplemented with 2% bovine serum albumin (Sigma) for another 30 min. Cells were then incubated overnight with primary antibody (1:200) in 10% normal goat serum (Sigma)/PBS. After rinsing, the coverslips were incubated with secondary antibodies FITC-conjugated (1:1000) for 4 h. All steps were performed at room temperature and in between incubation steps cells were rinsed with PBS. Cells were counterstained with 0.03% Evans blue/PBS. Coverslips were mounted on slides in Vectashield (Vector Laboratories, Burlingame, Calif.). Cells were examined using a Zeiss Axiophot microscope equipped with appropriate filters. Specificity of the immunolabeling was checked for by replacing the primary antibody with PBS.

RNAse protection assays. Total RNA was prepared with Tri reagent (MRC, Inc., Cincinnati, Ohio) according to the manufacturer's instructions. RNAse protection assays with 15 μg of RNA per reaction were carried out as described previously¹² using human probes for MHC class II (DR-α, CIITA, exon 1 of the promoter IV-specific form of CIITA (pIV-CIITA), and GAPDH as a control for RNA loading. Signal quantitation was determined using a phosphoimager analysis system (Bio-Rad, Hercules, Calif.). Levels of DR-α, CIITA, and pIV-CIITA RNA in any given sample were normalized to the GAPDH signal for that sample.

Western blots analysis. Cells were harvested in ice-cold RIPA solubilization buffer, and total amounts of protein were determined using a bicinchoninic acid quantification assay (Pierce, Rockford, Ill.) Fifty μg of total protein/lane were separated by SDS/PAGE under reducing conditions and blotted to polyvinylidene difluoride membranes (Millipore Corp., Bedford, Mass.) using a semidry blotting apparatus (Bio-Rad, Hercules, Calif.). Blots were blocked overnight in 5% defatted dry milk/PBS/0.1% Tween, and then incubated for 1 hour at room temperature with primary antibody (1:200) (mouse monoclonal anti-human p-Stat1α Santa Cruz, San Diego, Calif.), or mouse monoclonal anti-human β-actin (1:5000) (Pharmingen) for control of loading. This was followed by a 1 hour incubation with secondary peroxidase-conjugated antibody (1:10′000), (Jackson Immunoresearch, West Grove, Pa.). All steps were performed at room temperature and in between incubation steps cells were rinsed with PBS/0.1% Tween. Immunoreactivity was detected using the enhanced chemiluminescence detection method according to the manufacturer's instructions. (Amersham, Dübendorf, Switzerland), and subsequent exposure of the membranes to x-ray film.

Cytokine assay. Release of IL-2 from T lymphocytes was measured using ELISA kits, as suggested by the manufacturer (R&D, Abington, UK). Experiments were performed in the presence of polymyxin B (1 μg/mL). Antibody binding was detected by adding p-nitrophenyl phosphate (1.39 mg/mL), and absorbance was measured at 405 nm in a Dynatech plate reader. The amount of IL-2 detected was calculated from a standard curve prepared with human recombinant IL-2. Samples were assayed in triplicate.

### Results

As part of an exploration of possible interfaces between immune mechanisms and atherogenesis, and to evaluate possible beneficial effects of statins independently of their well-known effect as lipid lowering agents, the effect of statins on various features of the control of MHC class II expression and of subsequent lymphocyte activation has been analyzed.

The effect of several statins was studied on the regulation of both constitutive MHC class II expression in highly specialized antigen presenting cells (APC) and inducible MHC class II expression by interferon gamma (IFN-γ) in a variety of other cell types, including primary cultures of human endothelial cells (ECs) and monocyte-macrophages (Mφ).

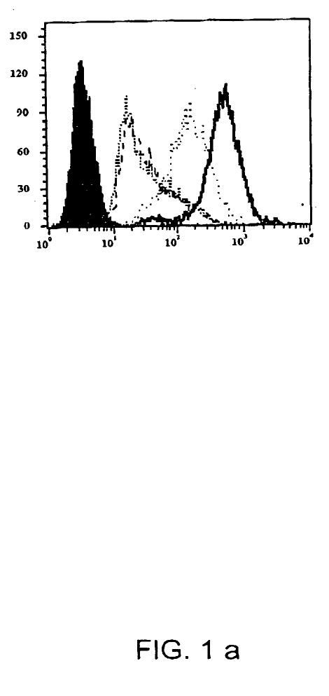

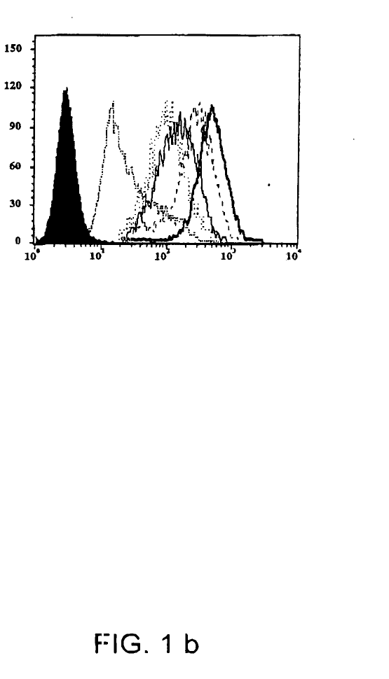

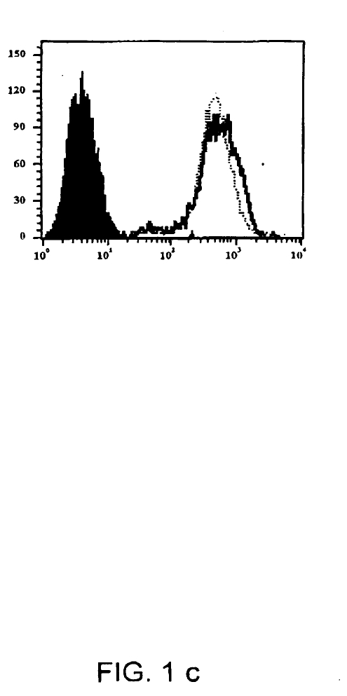

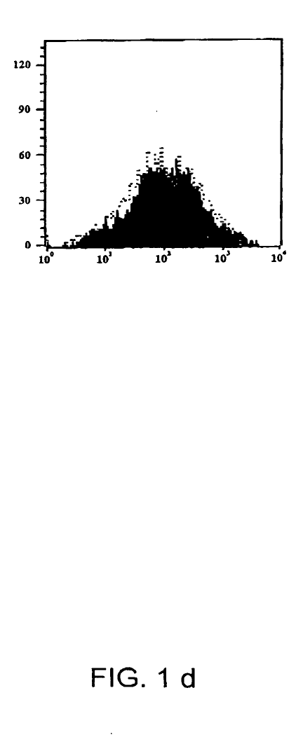

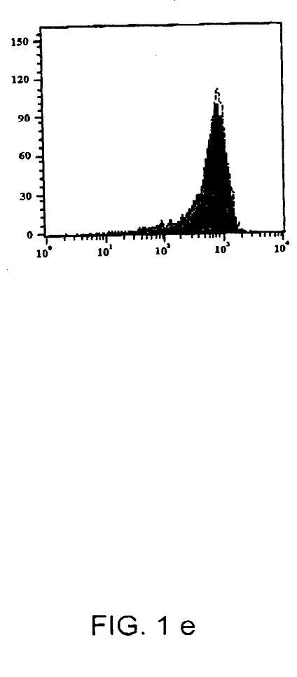

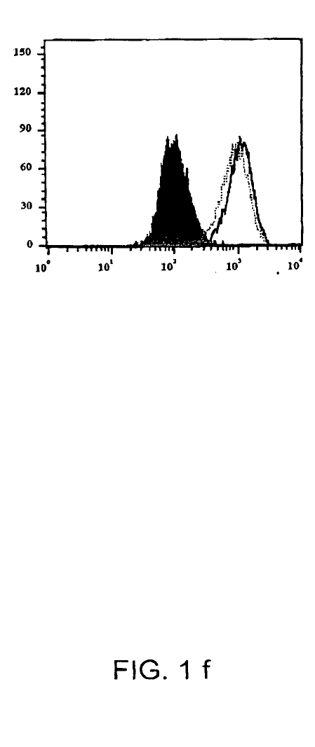

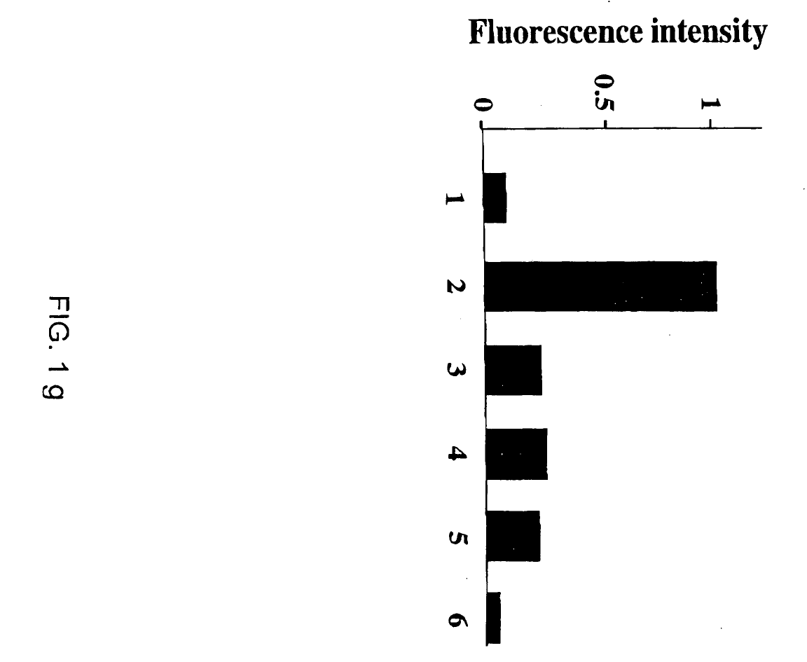

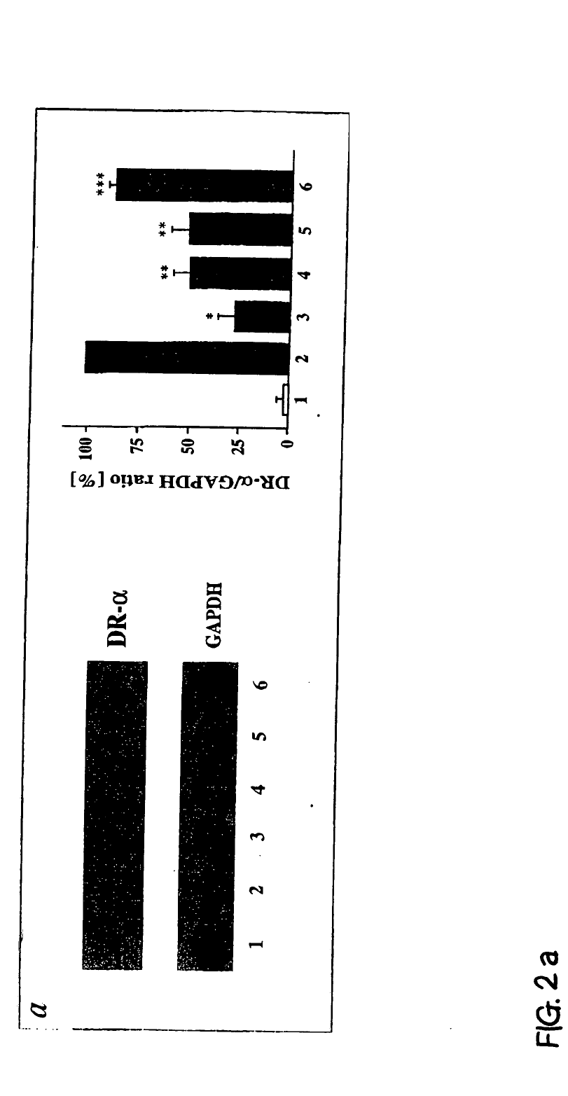

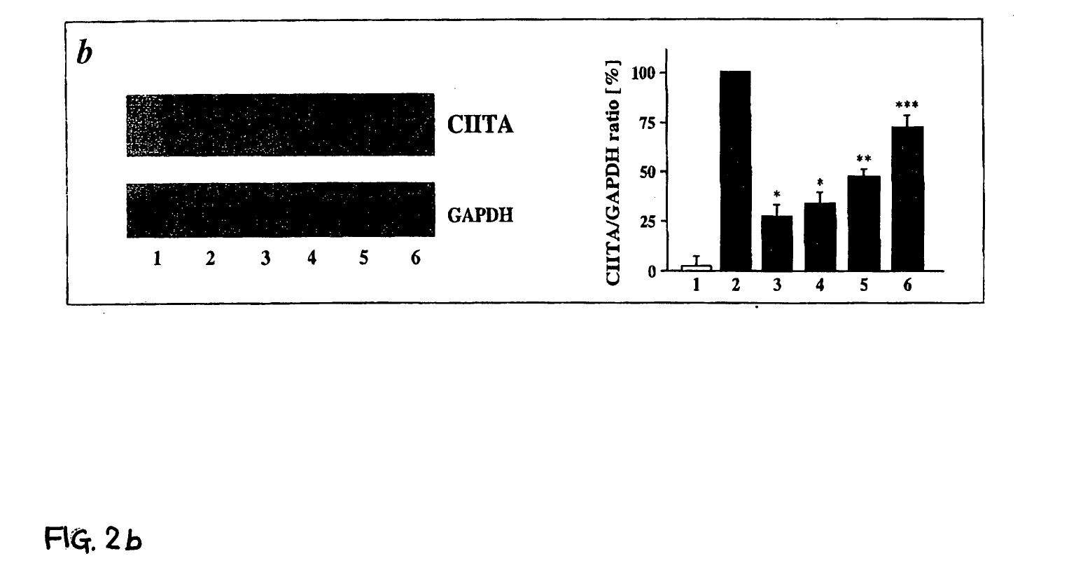

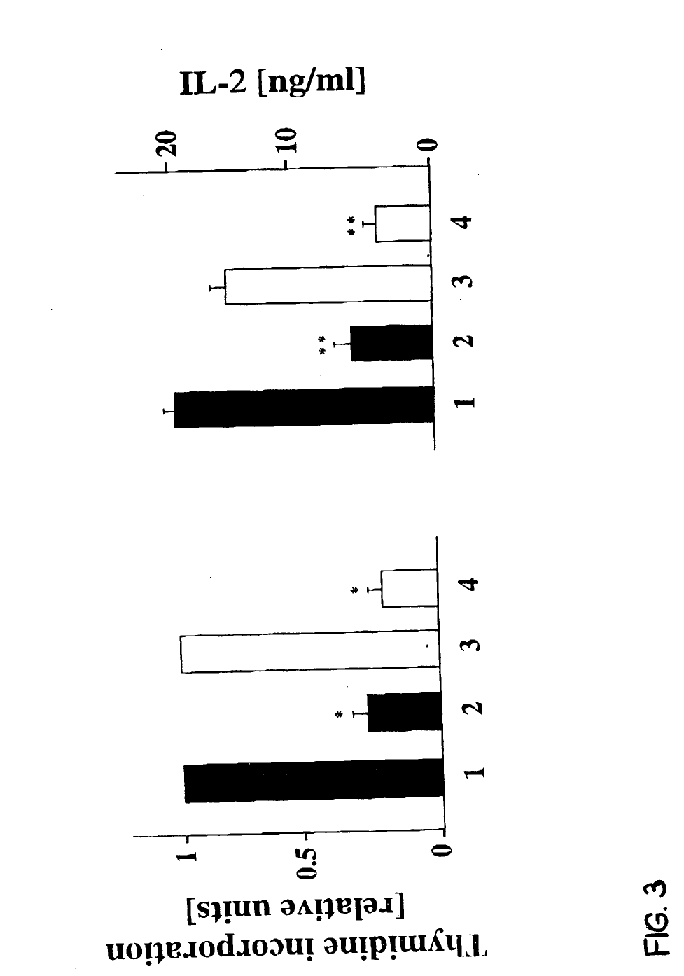

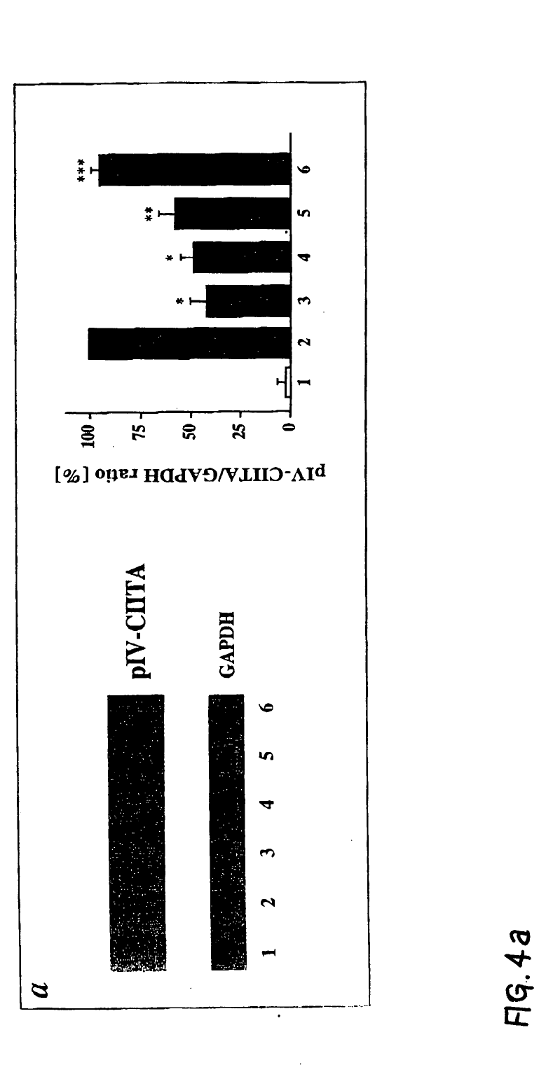

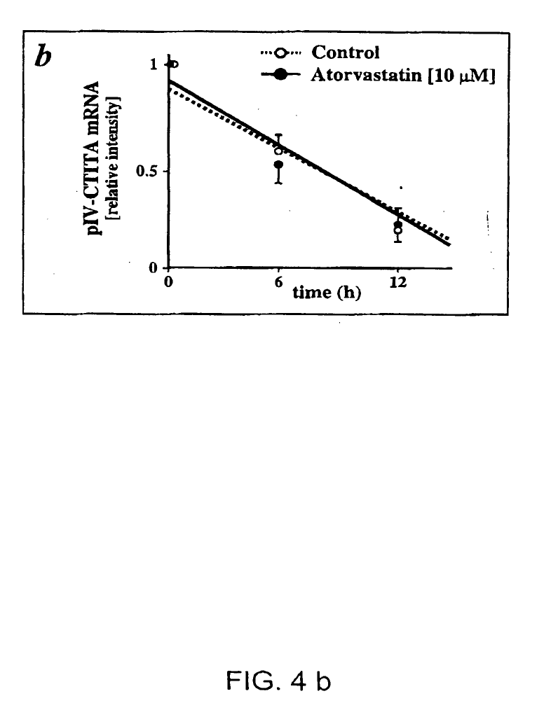

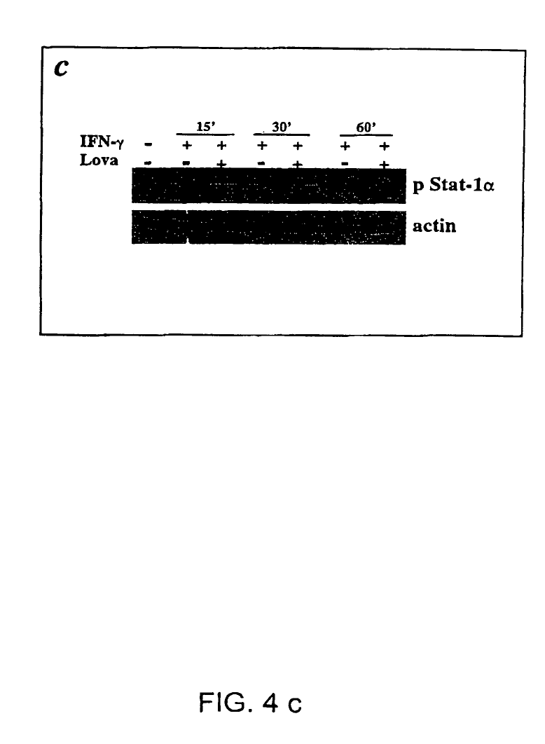

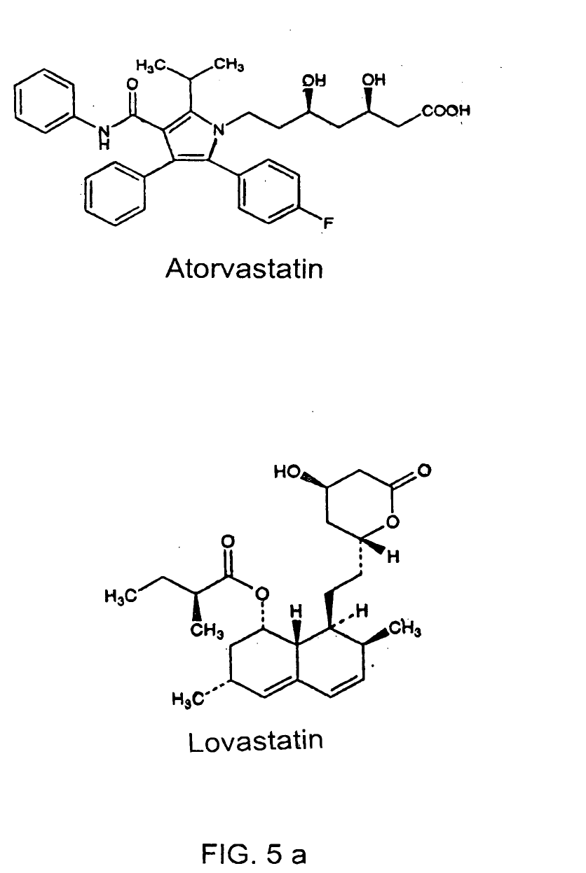

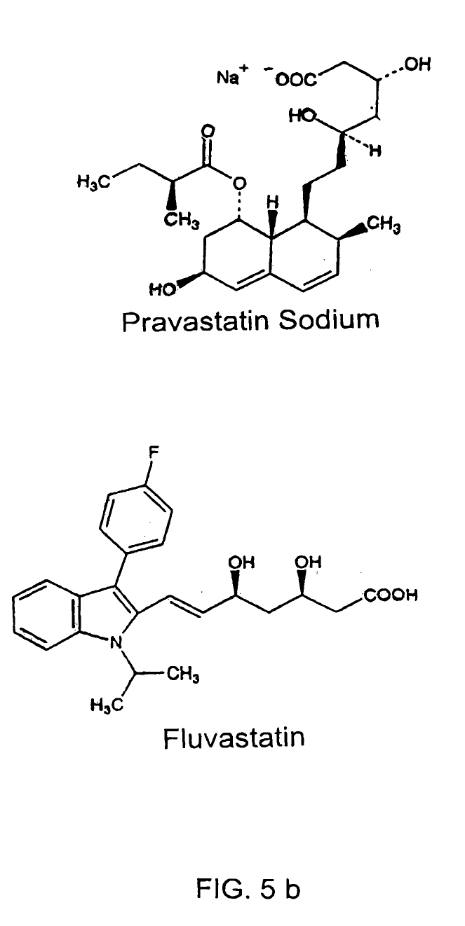

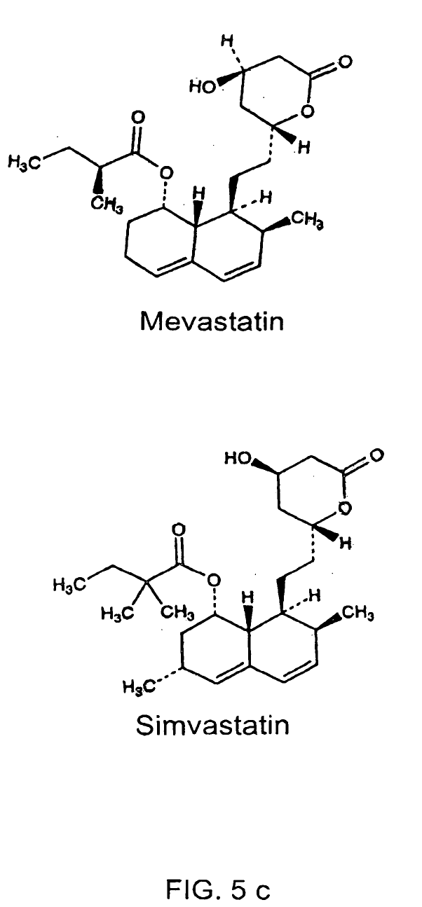

Experiments were performed to monitor cell surface expression (assayed both by FACS, FIG. 1a-f, and by immunofluorescence, FIG. 1g, as well as mRNA levels (RNAse protection assay, FIG. 2a) of MHC class II. These investigations have led to the following conclusions: 1) Statins effectively repress the induction of MHC-II expression by IFN-γ and do so in a dose-dependant manner (FIG. 1a-b, g). 2) In the presence of L-mevalonate, the effect of statins on MHC class II expression is abolished, indicating that it is indeed the effect of statins as HMG-CoA reductase inhibitors that mediates repression of MHC class II (FIG. 1c). 3) Interestingly, repression of MHC class II expression by statins is highly specific for the inducible form of MHC-II expression and does not concern constitutive expression of MHC-II in highly specialized APCs, such as dendritic cells and B lymphocytes (FIG. 1 d,e). 4) This effect of statins is specific for MHC class II and does not concern MHC class I expression (FIG. 1f). 5) In order to investigate functional implications of statin-induced inhibition of MHC class II expression, we performed mix lymphocyte reactions (allogenic T lymphocytes incubated with IFN-γ-pretreated human ECs or Mφ). T cell proliferation could be blocked by anti-MHC class II mAb (monoclonal antibody). Pretreatment of ECs or Mφ with statins represses induction of MHC class II and reduces subsequent T lymphocyte activation and proliferation measured by thymidine incorporation (FIG. 3a) or IL-2 release (FIG. 3b).

The novel effect of statins as MHC class II repressor was also observed and confirmed in other cell types, including primary human smooth muscle cells and fibroblasts, as well as in established cell lines such as ThP1, melanomas and Hela cells. This effect of statins on MHC class II induction is observed with different forms of statins currently used in clinical medicine. Interestingly however, different statins exhibit quite different potency as MHC class II <<repressors>> (see FIG. 1 a). Of the forms tested, the most powerful MHC class II repressor is Atorvastatin. The newly described effect on MHC class II repression can be optimized by screening other members of the statin family, as well as analogues of statins.

Repression of induction of MHC class II by IFN-γ, in statin treated samples, is paralleled by a reduced induction of CIITA mRNA by IFN-γ (FIG. 2 a,b), which points to an inhibition of induction of the CIITA gene by statins. Interestingly, the different degree of repression of CIITA mRNA induction observed with the different forms of statins (FIG. 2b) are reflected in the different levels of repression of MHC class II expression observed with the same drugs (FIG. 1a). This confirms the quantitative nature of the control of CIITA over MHC class II gene activity¹³. Constitutive expression of MHC class II, known to be mediated by CIITA promoters I and III, is not affected by statins (FIG. 1d,e), suggesting that promoter IV may be their sites of action. Indeed, we also show that induction of expression of the first exon specifically controlled by CIITA promoter IV is affected by statins (FIG. 4a). Finally, the statin effect is transcriptional, as demonstrated by actinomycin D experiments used to block de novo RNA synthesis and explore mRNA half-life (FIG. 4b), and it is direct and does not require de novo protein synthesis, as seen by a lack of effect of cycloheximide experiments.

As expected from the lack of statin effect on MHC class I induction (which is known to require Stat1α)¹⁴ the statin effect reported here is not due to an impairment of Stat1α activation, as phosphorylation and nuclear translocation of Stat1α occurs normally under the effect of statins (FIG. 4c).

### REFERENCES

1. Maron, D J., Fazio, S. & Linton, M. F. Current perspectives on statins. Circulation 101, 207-213 (2000).

2. Vaughan, C. J., Gotto, A. M. & Basson, C. T. The evolving role of statins in the management of atherosclerosis. J. Am. Coll. Cardiol. 35, 1-10 (2000).

3. Pedersen, T. R. Statin trials and goals of cholesterol-lowering therapy after AMI. Am. Heart. J. 138, 177-182 (1999).

4. Kobashigawa, J. A. et al. Effect of pravastatin on outcomes after cardiac transplantation. N. Engl. J. Med. 333, 621-627 (1995).

5. Mach, B., Steimle, V., Martinez-Soria, E. & Reith, W. Regulation of MHC class II genes: lessons from a disease. Annu. Rev. Immunol. 14, 301-331 (1996).

6. Steimle, V. et al. Complementation cloning of an MHC class II transactivator mutated in hereditary MHC class II deficiency (or bare lymphocyte syndrome) Cell 75, 135-146 (1993).

7. Steimle, V. et al. Regulation of MHC class II expression by interferon-gamma mediated by the transactivator gene CIITA. Science 265, 106-109 (1994).

8. Hebert, P. R., Gaziano, J. M., Chan, K. S. & Hennekens, C. H. Cholesterol lowering with statin drugs, risk of stroke, and total mortality. An overview of randomized trials. JAMA 278, 313-21 (1997).

9. Vaughan, C J., Murphy, M. B. & Buckley, B. M. Statins do more than just lower cholesterol. Lancet 348, 1079-1082 (1996).

10. Masternak, K. et al. A gene encoding a novel RFX-associated transactivator is mutated in the majority of MHC class II deficiency patients. Nat. Genet. 20, 273-277 (1998).

11. Muhlethaler-Mottet, A. et al. Expression of MHC Class II molecules in different cellular and functional compartments is controlled by differential usage of multiple promoters of transactivator CIITA. EMBO J. 16, 2851-2860 (1997).

12. Muhlethaler-Mottet, A. et al. Activation of MHC Class II transactivator CIITA by interferon gamma requires cooperative interaction between Stat1 and USF-1. Immunity 8, 157-166 (1998).

13. Otten, L. A., Steimle, V., Bontron, S. & Mach, B. Quantitation control of MHC Class II expression by the transactivator CIITA. Eur. J. Immunol. 82, 473-478 (1998).

14. Lee, Y. J., & Benveniste, E. N. Stat1 alpha expression is involved in IFN-gamma induction of the class II transactivator and class II MHC genes. J. Immunol. 157, 1559-1568 (1996).

15. Mach, F. et al. Functional CD40 is expressed on human vascular endothelial cells, smooth muscle cells, and macrophage: Implication for CD40-CD40 ligand signaling in atherosclerosis. Proc. Natl. Acad. Sci. USA. 94, 1931-1936 (1997).

16. Arrighi, J. F., Hauser, C., Chapuis, B., Zubler, R. H. & Kindler, V. Long-term culture of human CD34(+) progenitors with FLT3-ligand, thrombopoietin, and stem cell factor induces extensive amplification of a CD34(−)CD14(−) and a CD34(−)CD14(+) dendritic cell precursor. Blood 93, 2244-2252 (1999).

17. McPherson, R., Tsoukas, C, Baines, M. G., Vost, A., Melino, M. R., Zupkis, R. V. & Pross, H. F. Effects of lovastatin on natural killer cell function and other immunological parameters in man. J. Clin. Immunol. 13, 439-444 (1993).

18. Cutts, J. L. & Bankhurst, A. D. Suppression of lymphoid cell function in vitro by inhibition of 3-hydroxy-3-methylglutaryl coenzyme A reductase by lovastatin. Int. J. Immunopharmacol. 11, 863-869 (1989).

19. Roth, B. D., Bocan, T. M. A., Blankley, C. J., et al. Relation between tissue selectivity and lipophilicity for inhibitors of HMG-CoA reductase. J. Med. Chem. 34, 463-466 (1991).

20. Shaw, M. K., Newton, R. S., Sliskovic, D. R., Roth, B. D., Ferguson, E. & Krause, B. R. Hep-G2 cells and primary rat hepatocytes differ in their response to inhibitors of HMG-CoA reductase. Biochem. Biophys. Res. Commun. 170, 726-734 (1990).

1. A method to achieve MHC-class II mediated immunomodulation in a human with an MHC Class II-mediated inflammatory or autoimmune disorder characterized by IFN-γ inducible Class II transactivator expression, the method comprising administering to said human at least one statin selected from the group consisting of compactin, atorvastatin, lovastatin, pravastatin, fluvastatin, mevastatin, cerivastatin, and simvastatin, in an amount effective to modulate MHC class II expression in said human, wherein the MHC Class II-mediated inflammatory or autoimmune disorder characterized by IFN-γ inducible Class II transactivator expression is multiple sclerosis or rheumatoid arthritis.

2. A method to achieve MHC-class II mediated immunosuppression in a human with an MHC Class II-mediated inflammatory or autoimmune disorder characterized by IFN-γ inducible Class II transactivator expression, the method comprising administering to said human at least one statin selected from the group consisting of compactin, atorvastatin, lovastatin, pravastatin, fluvastatin, mevastatin, cerivastatin, and simvastatin, in an amount effective to suppress MHC class II expression in said human, wherein the MHC Class II-mediated inflammatory or autoimmune disorder characterized by IFN-γ inducible Class II transactivator expression is multiple sclerosis or rheumatoid arthritis.

3. A method to achieve MHC-class II mediated anti-inflammatory effect in a human with an MHC Class II-mediated inflammatory or autoimmune disorder characterized by IFN-γ inducible Class II transactivator expression, the method comprising administering to said human at least one statin selected from the group consisting of compactin, atorvastatin, lovastatin, pravastatin, fluvastatin, mevastatin, cerivastatin, and simvastatin, in an amount effective to suppress MHC class II expression in said human, wherein the MHC Class II-mediated inflammatory or autoimmune disorder characterized by IFN-γ inducible Class II transactivator expression is multiple sclerosis or rheumatoid arthritis.

4. The method of claims 1, 2 or 3, wherein said human does not suffer from hypercholesterolaemia.

5. The method of claims 1, 2 or 3, wherein said amount is effective to specifically modulate IFN-γ inducible MHC class II expression.

6. The method of claims 1, 2 or 3, wherein said statin is administered in the absence of any other immunosuppressive agents.

7. The method of claims 1, 2 or 3, wherein said amount is between about 10 to about 80 mg per day.

8. The method of claims 1, 2 or 3, wherein said amount is between about 20 to about 40 mg per day.

9. The method of claims 1, 2 or 3, wherein said administration comprises intralesional, intraperitoneal, intramuscular or intravenous injection; infusion; or topical, nasal, oral, ocular or optic delivery.

10. The method of claims 1, 2 or 3, wherein said administration is made daily.

11. The method of claim 2 or 3, wherein the immunosuppression or anti-inflammatory effect is the result of repression of T lymphocyte activation.

12. The method of claims 1, 2 or 3, wherein said MHC Class II-mediated inflammatory or autoimmune disorder characterized by IFN-γ inducible Class II transactivator expression is multiple sclerosis.

13. The method of claims 1, 2 or 3, wherein said MHC Class II-mediated inflammatory or autoimmune disorder characterized by IFN-γ inducible Class II transactivator expression is rheumatoid arthritis.
